(19) United States
(12) Patent Application Publication
(10) Pub. No.: US 2009/0182262 A1
(43) Pub. Date: Jul. 16, 2009

(54) VALVE ACTUATOR FOR CONTROLLING A MEDICAL FLUID

(76) Inventor: Donald Busby, Tampa, FL (US)

Correspondence Address: BAXTER HEALTHCARE CORPORATION, 1 BAXTER PARKWAY, DF2-2E, DEERFIELD, IL, 60015, US

(21) Appl. No.: 12/014,587

(22) Filed: Jan. 15, 2008

(51) Int. Cl. A61M 1/28; A61M 1/00; A61M 1/14

(52) U.S. Cl. 604/28; 604/30; 604/29

(57) ABSTRACT

A machine for controlling a medical fluid includes a base and an actuator comprising an electroactive polymer and disposed on the base. The actuator is configured for movement between an extended position and a contracted position. A valve head is disposed on the actuator and adapted to engage a flexible membrane of a cassette against a valve seat of the cassette when the actuator is extended to perform a valving function to control the flow of a fluid within the cassette

### BACKGROUND OF THE INVENTION

The present invention relates generally to dialysis systems and other medical fluid systems. More specifically, the present invention relates to a system with valves using actuators with an electroactive polymer.

Dialysis is used to remove waste products in patients with impaired kidney function. In hemodialysis, the patient's blood is pumped through a dialyzer to clean the blood and then return it to the body. Peritoneal dialysis periodically infuses sterile aqueous solution into the peritoneal cavity. This solution is called peritoneal dialysis solution, or dialysate. Diffusion and osmosis exchanges take place between the solution and the bloodstream across the natural body membranes. These exchanges remove the waste products that the kidneys normally excrete. Automated Peritoneal Dialysis (APD) is a popular form of peritoneal dialysis. APD uses a dialysis machine to automatically infuse, dwell, and drain peritoneal dialysis solution to and from the patient's peritoneal cavity. The dialysis machine uses multiple valves to control the flow of dialysis fluid through the machine and to and from the patient.

### SUMMARY

There are many embodiments of the present invention, of which only a few are described herein. The disclosure is intended to be descriptive but not limiting.

The device disclosed herein uses an electroactive polymer actuator to force sheeting against a valve seat. The electroactive polymer actuator may be used in place of the solenoids used in conventional dialysis systems and other medical fluid systems. The electroactive polymer actuators described herein are silent, efficient, produce almost no heat, have no moving parts and weigh less than existing solenoids.

In one aspect, a machine for controlling a medical fluid includes a base and an actuator comprising an electroactive polymer and disposed on the base. The actuator is configured for movement between an extended position and a contracted position. A valve head is disposed on the actuator and adapted to engage a flexible membrane of a cassette against a valve seat of the cassette when the actuator is extended to perform a valving function to control the flow of a fluid within the cassette.

In another aspect, a method of operating a dialysis machine includes providing a dialysis cassette. The dialysis cassette includes a housing configured and arranged to be placed in the machine, fluid pathways within the housing for routing a fluid, a valve assembly including a valve seat in fluid communication with the fluid pathways, and a flexible membrane attached to the housing. A dialysis machine is provided. The dialysis machine includes a base, an actuator including an electroactive polymer and disposed on the base, the actuator configured for movement between an extended position and a contracted position, and a valve head disposed on the actuator. The dialysis cassette is placed in the dialysis machine. The flow of the fluid though the valve assembly is controlled by engaging the valve head with the flexible membrane to dispose the flexible membrane against the valve seat to perform a valving function. Electrical power is provided to the actuator to contract the electroactive polymer so that the valve head disengages the flexible membrane from the valve seat to allow flow of fluid through the cassette.

Additional features and advantages of the present invention are described in, and will be apparent from, the following Detailed Description of the Invention and the figures.

### BRIEF DESCRIPTION OF THE FIGURES

FIG. 1 is perspective view of an embodiment of a dialysis machine.

FIG. 2 is a rear elevational view of one side of the cassette interface of the dialysis machine of FIG. 1.

FIG. 3 is an exploded perspective view of an embodiment of a dialysis cassette.

FIG. 4 is an elevational view of the other side of the dialysis cassette of FIG. 3.

FIG. 5A is a sectional view showing the actuator and valve assembly of the dialysis machine of FIG. 1 in a closed configuration.

FIG. 5B is a sectional view showing the actuator and valve assembly of the dialysis machine of FIG. 1 in an open configuration.

FIG. 6 is a sectional view showing the actuator and valve assembly of the dialysis machine of FIG. 1.

FIG. 7 is a schematic view of an embodiment of a control system for the actuator of FIGS. 5A and 5B.

FIG. 8 is a perspective view of an embodiment of an electroactive polymer actuator.

FIG. 9 is a perspective view of another embodiment of an electroactive polymer actuator.

FIG. 10A is a sectional view showing an embodiment of an actuator and valve assembly in a closed configuration.

FIG. 10B is a sectional view showing an embodiment of an actuator and valve assembly in an open configuration.

### DETAILED DESCRIPTION

There are many embodiments of the present invention, of which only a few are described herein. The disclosure is intended to be descriptive but not limiting.

Existing dialysis and other medical fluid instruments utilize cassette technology to transfer fluids to and from the patient. The cassette uses a thin flexible plastic membrane to isolate the fluids from the instrument when the cassette is inserted into the instrument. To control fluid flow, valve seats are incorporated into the cassette. Existing dialysis instruments use electrical solenoids or pneumatic air pressure to force the membrane against the valve seat, thereby stopping fluid flow. The device disclosed herein uses an electroactive polymer actuator to force the membrane against the valve seat. An electroactive polymer is an elastomer that changes shape when electrical energy is applied to it. The electroactive polymer converts electrical energy to mechanical energy.

The electroactive polymer actuator is used in place of the mechanical solenoids or pneumatics and pneumatic solenoids used in conventional dialysis and other medical fluid systems. A solenoid converts electrical energy to mechanical energy when the solenoid is energized and the solenoid core moves axially with a certain force. Solenoids tend to produce a lot of heat, consume power, are noisy, contain multiple moving parts, and are heavy due to the materials of construction. The electroactive polymer actuators described herein are silent, efficient, produce almost no heat, have no moving parts, and have approximately 20% of the weight of existing solenoids.

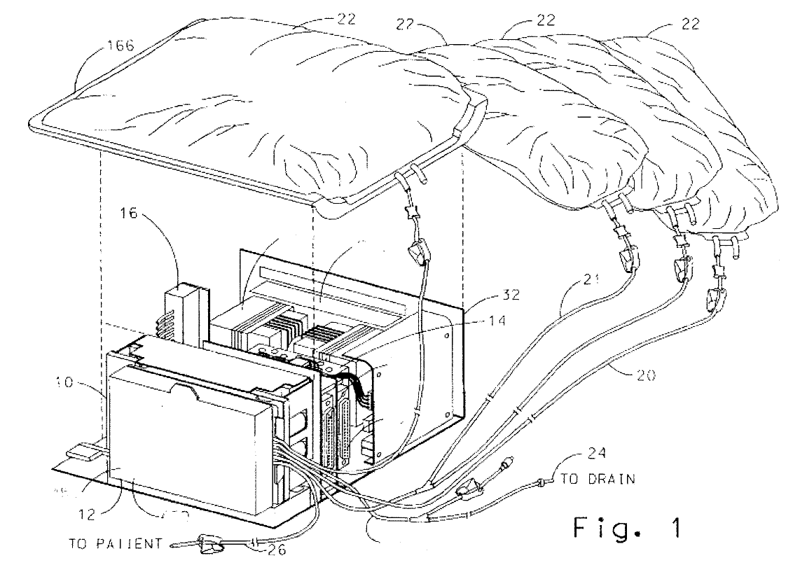

Referring now to the drawings, and especially to FIG. 1, an automated peritoneal dialysis (APD) system is generally shown therein and identified by numeral 10. The APD system 10 includes an automated peritoneal dialysis apparatus or cycler 12 comprising an electrical controller 14 coupled to supply control and drive signals to a distribution system 16. APD systems are well known in the art and are described, for example, in commonly owned U.S. Pat. No. 5,938,634, entitled “PERITONEAL DIALYSIS SYSTEM WITH VARIABLE PRESSURE DRIVE,” the teachings of which are incorporated herein by reference, and U.S. Pat. No. 5,350,357, entitled “PERITONEAL DIALYSIS SYSTEMS EMPLOYING A LIQUID DISTRIBUTION AND PUMPING CASSETTE THAT EMULATES GRAVITY FLOW. However, the system disclosed herein is not limited to use with such a system, but may be used with any type of dialysis system, including hemodialysis systems, and other types of medical fluid systems.

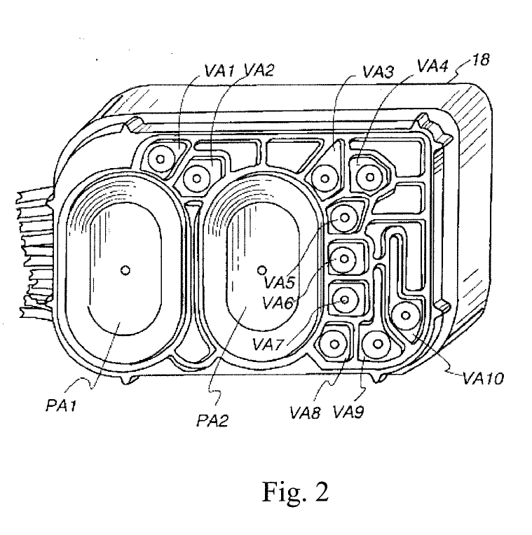

A disposable liquid or dialysate delivery system 20 includes a disposable liquid or dialysate delivery set 21 which may be coupled to the cassette interface 18 (shown in FIG. 2). A plurality of dialysate bags 22 may be connected to the disposable dialysate delivery set 21 for supply of dialysate through the disposable dialysate delivery set 21. The delivery set 21 includes a disposable dialysate cassette 23 for connection to a dialysate drain 24 and through a patient tube 26 to a catheter connected to a patient (not shown).

The cycler 12 has a housing 32 which holds the electrical controller 14, the supply system and the cassette interface 18. The controller 14 receives alternating current electric power from a suitable source. The set 21 is intended to be a single use, disposable item. The user loads the set 21 on the cycler 12 before beginning each APD therapy session. The user removes the set 21 from the cycler 12 upon the completing the therapy session and discards it.

As FIG. 3 best shows, the set 21 includes a cassette 23 to which lengths of flexible plastic tubes are attached. In use, the cassette 23 mounts inside a holder in the cycler 14. The cassette 23 serves in association with the cycler 12 and the controller 16 to direct liquid flow among the multiple liquid sources and destinations that a typical APD procedure requires. The cassette 23 provides centralized valving and pumping functions in carrying out the selected APD therapy.

FIG. 3 shows the details of the cassette 23. The cassette 23 includes a body having front and back sides 58 and 60. For the purposes of description, the front side 58 is the side of the cassette 23 that, when the cassette 23 is mounted in the holder, faces away from the user. A flexible membrane 59 and 61 overlies the front side and back sides 58 and 60 of the cassette 23, respectively. The cassette 23 is preferably made of a rigid medical grade plastic material. The flexible membranes 59, 61 are preferably made of flexible sheets of medical grade plastic. The flexible membranes 59, 61 are sealed about their peripheries to the peripheral edges of the front and back sides 58, 60 of the cassette 23.

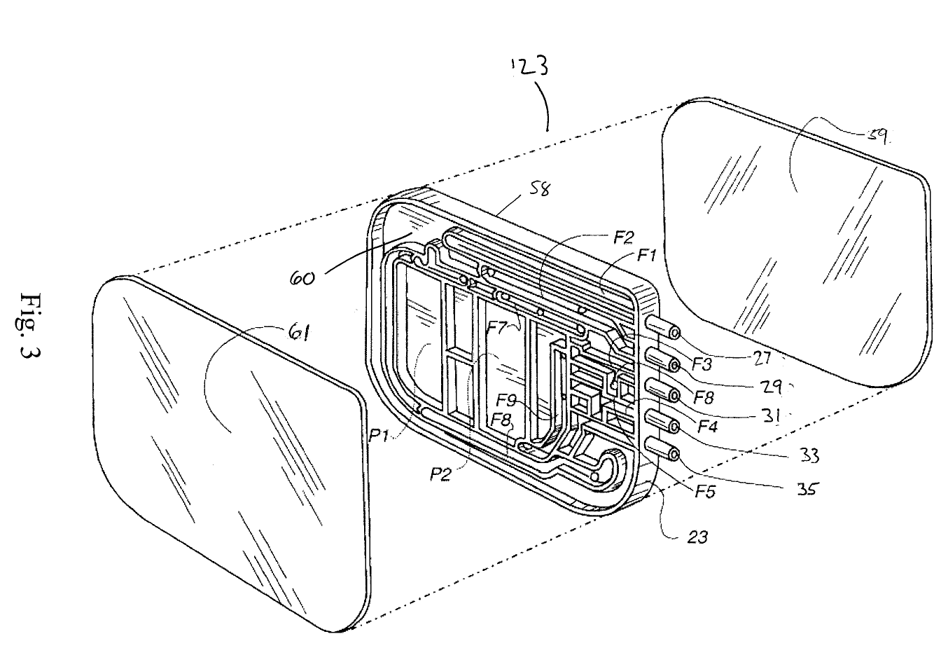

The cassette 23 forms an array of interior cavities in the shapes of wells and channels. The interior cavities create multiple pump chambers P1 and P2 (visible from the front side 58 of the cassette 23, as shown in FIG. 4). The interior cavities also create multiple paths F1 to F9 to convey liquid (visible from the back side 60 of the cassette 23, as FIG. 3 shows). The interior cavities create multiple valve stations V1 to V10 (visible from the front side 58 of the cassette 23, as FIG. 4 shows). The valve stations V1 to V10 interconnect the multiple liquid paths F1 to F9 with the pump chambers P1 and P2 and with each other. The number and arrangement of the pump chambers, liquid paths, and valve stations can vary.

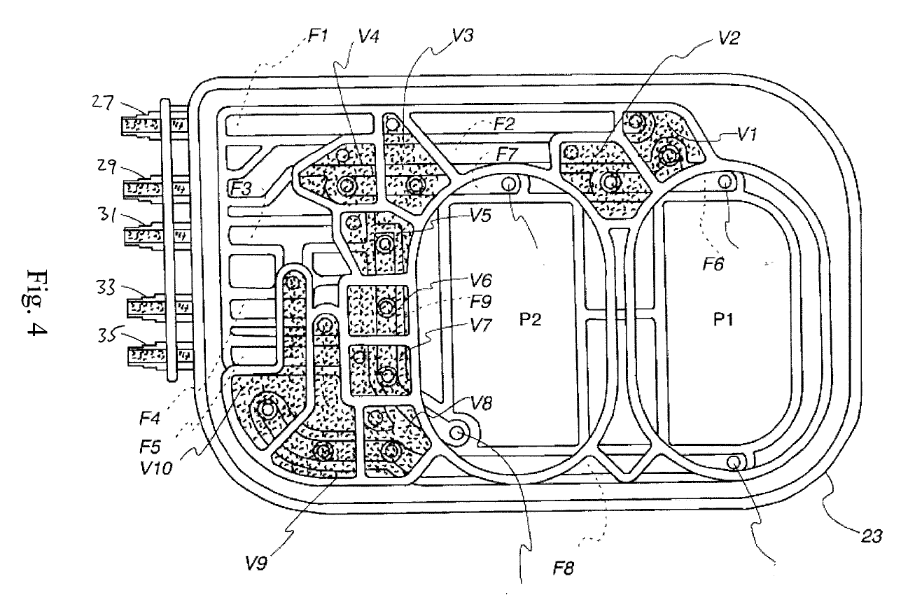

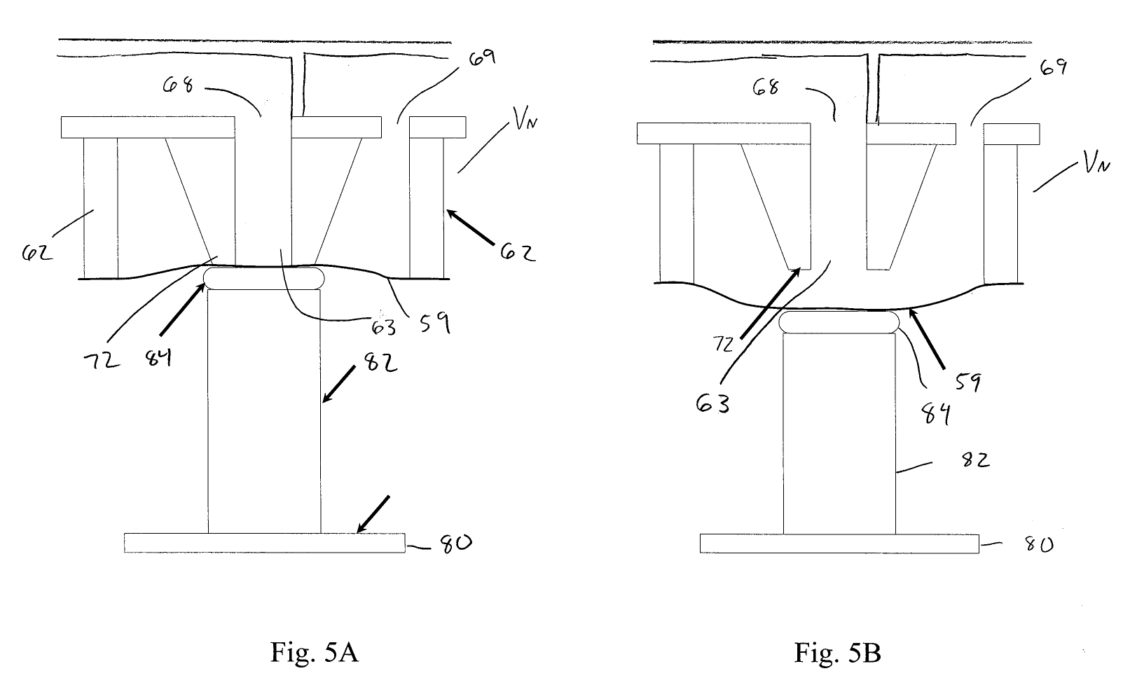

The ten valve stations V1 to V10 are formed as wells open on the front side 58 of the cassette 23. FIGS. 5A and 5B shows a typical valve station V_{N} disposed against an actuator 82. Upstanding edges 62 peripherally surround the open wells of the valve stations V1 to V10 on the front side 58 of the cassette 23. As FIGS. 5A and 5B best show, the valve stations V1 to V10 are closed on the back side 60 of the cassette 23, except that each valve station includes a pair of through holes or ports 68 and 69. One port 68 communicates with a selected liquid path on the back side 60 of the cassette 23. The other port 69 communicates with another selected liquid path on the back side 60 of the cassette 23.

In each valve station V1-V10, a raised valve seat 72 surrounds one of the ports 68. As FIG. 5B best shows, the valve seat 72 terminates higher than the surrounding peripheral edges 62. The other port 69 is flush with the front side 58 of the cassette. The flexible membrane 59 overlying the front side 58 of the cassette 23 rests against the upstanding peripheral edges 62 surrounding the pump chambers and valve stations. With the application of positive force uniformly against this side 58 of the cassette 23, the flexible membrane 59 seats against the upstanding edges 62. The positive force forms peripheral seals about the pump chambers P1 and P2 and valve stations V1 to V10. This, in turn, isolates the pump chambers P1 and P2 and valve stations V1 to V10 from each other and the rest of the system. As will be described below, the cycler 12 controls actuators 82 to apply positive force to the front cassette side 58.

Further localized application of positive and negative forces upon the regions of the flexible membrane 59 overlying these peripherally sealed areas serve to flex the flexible membrane regions within these peripherally sealed areas. These localized applications of positive and negative forces on the flexible membrane regions overlying the pump chambers P1 and P2 serve to move liquid out of and into the chambers P1 and P2. Likewise, these localized applications of positive and negative forces on the flexible membrane regions overlying the valve stations V1 to V10 will serve to seat and unseat these flexible membrane regions against the valve seats 72, thereby closing and opening the associated valve port 68.

In operation, the cycler 12 controls valve actuators 82 for opening and closing the valve ports. As shown in FIG. 4, the liquid paths F1 to F9 are formed as elongated channels that are open on the back side 60 of the cassette 23. Upstanding edges 62 peripherally surround the open channels on the back side 60 of the cassette 23. The liquid paths F1 to F9 are closed on the front side 58 of the cassette 23, except where the channels cross over valve station ports 68 and 69 or pump chamber ports.

As FIG. 3 shows, five premolded tube connectors 27, 29, 31, 33, and 35 extend out along one side edge of the cassette 23. When the cassette 23 is vertically oriented for use, the tube connectors 27 to 35 are vertically stacked one above the other. The first tube connector 27 is the uppermost connector, and the fifth tube connector 35 is the lowermost connector. As shown in FIG. 2, the cassette interface 18 includes pump actuators PA1 and PA2 and valve actuators VA1 to VA10. In the operating position, the two pump actuators PA1 and PA2 register with the two pump chambers P1 and P2 in the cassette 23. The ten valve actuators VA1 to VA10 likewise register with the ten valve stations V1 to V10 in the cassette 23.

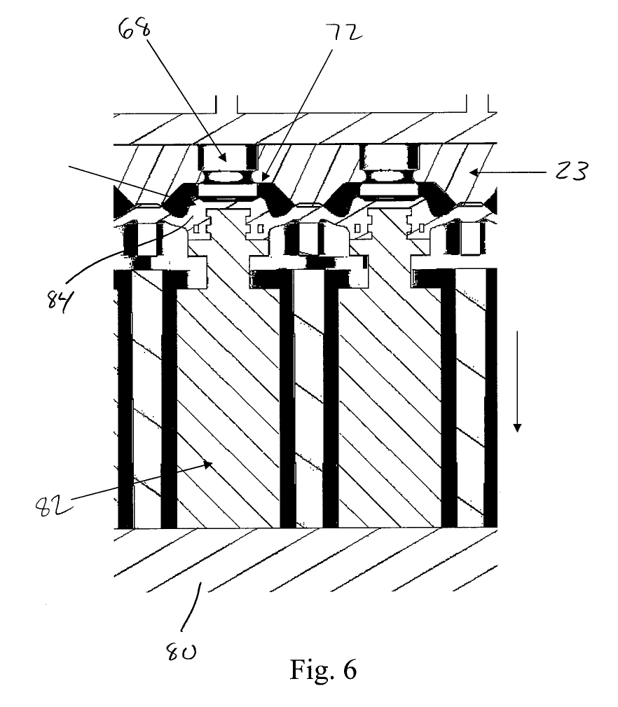

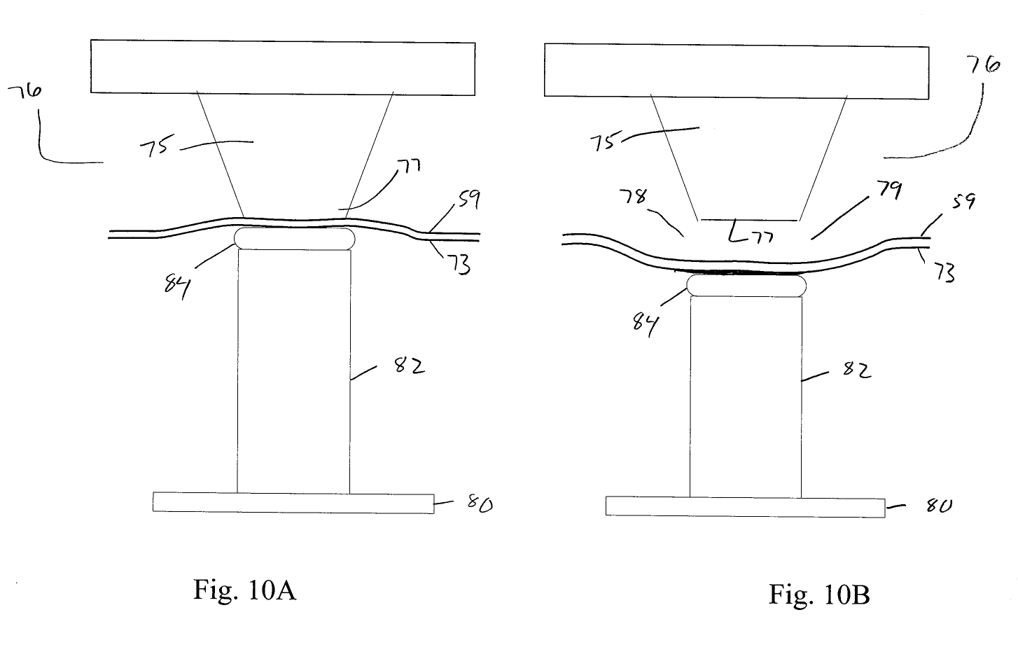

As previously described, the actuators VA1 to VA10 in the dialysis machine interact with the valve assemblies 63 in the dialysis cassette 23 to control the flow of dialysis fluid, other renal therapy fluid, or blood through the cassette 23. Turning now to the arrangement of the valve assemblies 63 and actuators 82, best seen in FIGS. 5A, 5B, and 6, the actuator 82 includes an electroactive polymer and is disposed on the base 80 of the interface 18. Although only one valve actuator is shown, it is to be understood that some or all of the valve actuators VA1 to VA10 may include the actuator design 82. The actuator 82 is configured for movement between an extended configuration (shown in FIG. 5A) and a contracted configuration (shown in FIG. 5B). A valve head 84 is disposed on the actuator 82 and is configured to engage the flexible membrane 59 of the cassette 23 against the valve seat 72 of the cassette 23 to perform a valving function in the cassette 23. The valving function prevents flow between fluid inlet 68 and a fluid outlet 69 to control the flow of the fluid within the cassette 23. Although a particular valve design is shown in FIGS. 5A, 5B, and 6, other valve designs are possible, such as that shown in FIGS. 10A and 10B below.

The electroactive polymer in actuator 82 converts electrical energy to mechanical energy. In the actuator design disclosed herein, the electrical energy is converted to linear force, thus moving the actuator 82 in the desired direction between an extended position and a contracted position. The electroactive polymer acts as a compliant capacitor, where a passive elastomer film is sandwiched between two electrodes. When a voltage U is applied, an electrostatic pressure pₑₗ arises from the Coulomb forces acting between the electrodes, thus squeezing the elastomer. The equivalent electromechanical pressure p_{eq} is twice the electrostatic pressure pₑₗ and is given by the following equation: p_{eq}=ε₀εᵣU²/z², where ε₀ is the vacuum permittivity, εᵣ is the dielectric constant of the polymer and z is the thickness of the elastomer film.

The polymer may assume a variety of configurations in the actuator 82. In one embodiment, shown in FIG. 8, a polymer is formed as a thin, flat sheet 92 that is rolled up in a generally cylindrical shape to form a cylindrical actuator 90. Electrodes 91, 93 provide a voltage to the actuator 90. In another embodiment, the polymer is formed into a plurality of thin, flat disks 98 that are disposed on top of each other to form a cylindrical actuator 96, as shown in FIG. 9. Electrodes 97, 99 provide a voltage to the actuator 96. The configuration of the polymer may depend on the stroke and force requirements of the actuator. For example, the polymer may also be shaped as a square, rectangle, oval, or as a diaphragm. The electrodes may be of any shape and material provided that they are able to supply a suitable voltage to the electroactive polymer.

FIG. 5A depicts the actuator in the unpowered state. With a cassette inserted into the instrument, there is a designed interference between the cassette valve assembly 63 and the actuator 82 such that a force of about 1 to 2 lbs, preferably 1.2-1.5 lbs is generated at the valve head 84 to seal the valve assembly 63 at fluid pressures of approximately 8 psi. In this configuration, there is no fluid flow. When voltage is applied to the actuator 82, as shown in FIG. 5B, the electroactive polymer changes shape, such that the length of the actuator 82 becomes shorter. The polymer material is displaced radially, so the actuator 82 becomes slightly larger in diameter. The actuator 82 is fixed to the instrument base or base 80, so as the actuator 82 becomes shorter, the valve head 84 retracts from the membrane 59 and valve seat 72, and fluid is allowed to flow through the valve assembly 63.

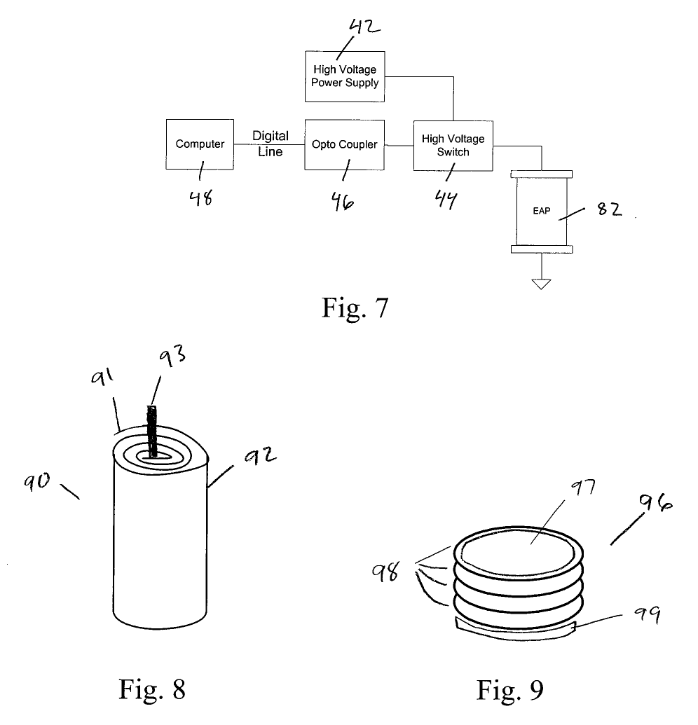

FIG. 7 denotes a typical driver circuit for the actuators. A high voltage power supply 42 is in electrical connection with the actuator 82. A high voltage switch 44 is in electrical communication between the high voltage power supply 42 and the actuator 82. The high voltage switch 44 is controlled by a computer 48 through an opto-coupler 46. The voltage to drive the electroactive polymer is in the range of 2000 to 4000 volts, with 2000 volts being typical. Although the voltage is high, the current is very low, on the order of about 1 milliamp. Because the current is so low, the actuators 82 generate almost no heat, which allows them to be packaged very close to each other when multiple actuators are used. The actuators 82 could be individually wired with two small leads to the electrodes, the leads then attached to a circuit board via a connector. The leads might also be a ribbon cable or flex circuit. An array of actuators 82 may be directly soldered to a circuit board.

Due to the properties of the electroactive polymer, not only can electrical energy be translated to mechanical energy (in this case, linear motion), but additionally, mechanical energy can be converted back to electrical energy. As the electroactive polymer is compressed mechanically, the polymer generates an output voltage. By measuring specific attributes of this electrical energy, it is possible to determine the position of the valve head 84 and the force applied to the valve seat 72. Thus, the system may include a positional measurement system in communication with the actuator 82.

An alternative embodiment of a valve assembly 76 is shown in FIGS. 10A and 10B. The actuator 82 is the same as that shown in FIGS. 5A and 5B and is configured for movement between an extended configuration (shown in FIG. 10A) and a contracted configuration (shown in FIG. 10B). The valve assembly 76 includes an extending portion 75 with a valve seat 77 on the distal end. Flexible membrane 59 may include a conducting portion 73 that serves as an electrode for the electroactive polymer of the actuator 82. The conducting portion 73 may cover all or only a portion or portions of membrane 59. If conducting portion 73 is used, then valve head 84 may also be made of a conducting material. The valve head 84 is configured to engage the flexible membrane 59 of the cassette 23 against the valve seat 77 to perform a valving function. The valving function prevents flow between fluid inlet 78 and a fluid outlet 79 to control the flow of the fluid.

As is the case with many disposable medical products, the dialysis cassette and many of the parts are preferably made from a medical-grade plastic. In particular, injection-molded parts are preferred, because they can be produced in high volume at low cost with excellent materials. Other methods of making may also be used, such as thermoforming, extruding, compression molding, thermoforming, blow molding, and the like. The valve head may be made of plastic or metal, depending on the configuration of the actuator.

Materials suitable for use as an electroactive polymer may include any substantially insulating polymer or elastomer that deforms in response to an electrostatic force or whose deformation results in a change in electric field. Exemplary materials suitable for use include silicone elastomers, acrylic elastomers, polyurethanes, thermoplastic elastomers, and the like.

Materials used as an electroactive polymer may be selected based on one or more material properties such as high electrical breakdown strength, a low modulus of elasticity, a high dielectric constant, or the like. The material preferably has a low stiffness, a high dielectric constant, and high electrical breakdown strength. In one embodiment, the polymer has an elastic modulus of less than about 100 MPa. In another embodiment, the polymer has a maximum actuation pressure between about 0.05 MPa and about 10 MPa, and preferably between about 0.3 MPa and about 3 MPa. In another embodiment, the polymer has a dielectric constant between about 2 and about 20, and preferably between about 2.5 and about 12. The present disclosure is not intended to be limited to these ranges. In many cases, electroactive polymers may be fabricated and implemented as thin films. Thicknesses suitable for these thin films may be below 50 micrometers.

As electroactive polymers may deflect at high strains, the electrodes attached to the polymers should also deflect without compromising mechanical or electrical performance. Generally, electrodes suitable for use may be of any shape and material provided that they are able to supply a suitable voltage to the electroactive polymer. In one embodiment, the electrodes adhere to a surface of the polymer. Electrodes adhering to the polymer are preferably compliant to conform to the changing shape of the polymer. The electrodes may be applied to only a portion of an electroactive polymer. Various types of electrodes suitable for use with the present disclosure include structured electrodes comprising metal traces and charge distribution layers, textured electrodes, conductive greases, colloidal suspensions, high aspect ratio conductive materials such as carbon fibrils and carbon nanotubes, and mixtures of conductive materials.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

What is claimed is:

1. A machine for controlling flow of a medical fluid comprising:
a base;
an actuator comprising an electroactive polymer and disposed on the base, the actuator configured for movement between an extended position and a contracted position; and
a valve head disposed on the actuator, the valve head adapted to engage a flexible membrane of a cassette against a valve seat of the cassette when the actuator is extended to perform a valving function to control the flow of a fluid within the cassette.

2. The machine of claim 1 further comprising a high voltage power supply in electrical connection with the actuator.

3. The machine of claim 2 further comprising a high voltage switch in electrical communication between the high voltage power supply and the actuator.

4. The machine of claim 2 further comprising electrodes disposed on the actuator and in electrical communication between the high voltage power supply and the actuator.

5. The machine of claim 1 wherein the electroactive polymer is selected from silicone elastomers, acrylic elastomers, polyurethanes, and combination thereof.

6. The machine of claim 1 wherein the actuator comprises a plurality of disks of electroactive polymer disposed atop each other.

7. The machine of claim 1 wherein the actuator comprises a sheet of electroactive polymer rolled up in the shape of a cylinder.

8. The machine of claim 1 further comprising a positional measurement system in communication with the actuator.

9. The machine of claim 1 further comprising electrodes in electrical communication with the actuator.

10. The machine of claim 9 wherein the electrodes are compliant.

11. The machine of claim 1 wherein the actuator provides a closed valve position when there is no electrical current applied to the actuator.

12. The machine of claim 1 wherein the actuator provides a force of 1 to 2 lbs between the valve head and the valve seat.

13. The machine of claim 1 wherein the dialysis machine comprises a cassette interface and the actuator is mounted on the cassette interface.

14. A dialysis machine assembly comprising:
a dialysis cassette comprising:
a housing;
fluid pathways within the housing for routing a fluid;
a valve assembly in fluid communication with the fluid pathways, comprising:
a fluid inlet;
a fluid outlet;
a valve seat in fluid communication with the fluid inlet and the fluid outlet; and
a flexible membrane attached to the housing, the membrane and valve assembly cooperating with the machine to perform a valving function controlling flow of the fluid though the valve assembly; and
a dialysis machine configured to hold the dialysis cassette comprising:
a base;
an actuator comprising an electroactive polymer and disposed on the base, the actuator configured for movement between an extended position and a contracted position; and
a valve head disposed on the actuator, the valve head adapted to engage the flexible membrane against the valve seat when the actuator is extended to perform the valving function between the fluid inlet and the fluid outlet.

15. The dialysis machine assembly of claim 14 wherein the valve assembly comprises upstanding peripheral edges, wherein the valve seat is disposed at a distal end of the upstanding peripheral edges and the fluid inlet is disposed at a proximal end of the upstanding peripheral edges.

16. The dialysis machine assembly of claim 14 wherein the flexible membrane comprises a conducting portion to serve as an electrode for the actuator.

17. A method of operating a dialysis machine comprising:
providing a dialysis cassette comprising:
a housing configured and arranged to be placed in the machine;
fluid pathways within the housing for routing a fluid;
a valve assembly comprising a valve seat in fluid communication with the fluid pathways; and
a flexible membrane attached to the housing;
providing a dialysis machine comprising:
a base;
an actuator comprising an electroactive polymer and disposed on the base, the actuator configured for movement between an extended position and a contracted position; and
a valve head disposed on the actuator;
placing the dialysis cassette in the dialysis machine;
controlling flow of the fluid though the valve assembly by engaging the valve head with the flexible membrane to dispose the flexible membrane against the valve seat to perform a valving function;
providing electrical power to the actuator to contract the electroactive polymer so that the valve head disengages the flexible membrane from the valve seat to allow flow of fluid through the cassette.

18. The method of operating a dialysis machine of claim 17 further comprising determining the position of the actuator by measuring the electrical properties of the electroactive polymer.

19. The method of operating a dialysis machine of claim 17 further comprising determining the force experienced by the actuator by measuring the electrical properties of the electroactive polymer.
